(19) United States
(12) Patent Application Publication
(10) Pub. No.: US 2006/0253026 A1
(43) Pub. Date: Nov. 9, 2006

(54) Transducer for multi-purpose ultrasound

(75) Inventors: Wayne Gueck, Redmond, WA (US); Xuan-Ming Lu, San Jose, CA (US)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Medical Solutions USA, Inc.

(21) Appl. No.: 11/123,585

(22) Filed: May 4, 2005

(51) Int. Cl. A61B 8/00

(52) U.S. Cl. 600/439; 600/437

(57) ABSTRACT

Multiple transducer layers are provided for one or more elements of a transducer array. One layer is used for imaging, and another layer is used for generating acoustic energy for other purposes. Since the two layers of an element are used for different purposes, separate cables are provided for transmitting different electrical signals. Alternatively, a switch or other mechanism is provided for sequentially routing different signals to the different layers. In another transducer, elements distributed along an azimuth dimension of the transducer array have different numbers of transducer layers. Elements associated with a fewer number of layers are used for higher frequency signals, and elements with a greater number of layers are used for lower frequency signals.

### BACKGROUND

The present invention relates to a transducer for multiple purpose ultrasound. In particular, a transducer is provided for both ultrasound imaging and other ultrasound purposes.

In addition to imaging, ultrasound may be used to create strain through remote palpitation, create streaming of fluids within masses or cysts, create shear waves, move contrast agents or microbubbles, break or destroy microbubbles, heat tissue or other purposes. Designing an ultrasound transducer for both imaging as well as other purposes may be difficult. The other purposes may demand greater transmit power at one frequency, and the imaging may demand a wide bandwidth at another frequency. For example, 1 to 2 MHz frequency for one purpose and a 4-5 MHz or higher frequency for an imaging purpose. A transducer with limited bandwidth may not operate efficiently for both applications.

### BRIEF SUMMARY

By way of introduction, the preferred embodiments described below include multi-purpose transducers and methods for using a transducer for multiple purposes. In one embodiment, multiple transducer layers are provided for one or more elements of the transducer. One layer is used for imaging, and another layer is used for generating acoustic energy for other purposes. Since the two layers of an element are used for different purposes, separate cables are provided for transmitting different electrical signals. Alternatively, a switch or other mechanism is provided for sequentially routing different signals to the different layers. In another embodiment, elements distributed along an azimuth dimension of the transducer have different numbers of transducer layers. Elements associated with a fewer number of layers are used for higher frequency signals and elements with a greater number of layers are used for lower frequency signals. In another embodiment, elements distributed along an elevation dimension of the transducer have different numbers of layers. In this case, a first layer may be a 1.x-D or 2-D array while the other layer is only a 1-D array, or, a 1.y-D array having a lesser number of elevation rows than the first layer. In another embodiment, the first layer and second layer can be both a 1.x-D or 2D array with same or different pitch in elevation and/or azimuth. These embodiments may be used separately or in combination.

In a first aspect, an ultrasound transducer for multi-purpose use is provided. A first transducer layer of an element is operable for transmission and reception. A second transducer layer of the same element is operable only for transmission and not for reception. The transducer layers are distributed in range. A cable connects with an electrode of the first layer. Another cable connects with an electrode of the second transducer layer.

In a second aspect, a method is provided for using a transducer to image and apply other acoustic energy. Acoustic energy is transmitted with a first transducer layer of an element of the transducer. The transmission is provided in response to a first electrical signal. The first transducer layer receives acoustic echoes in response to the transmission. A second transducer layer of the element of the transducer transmits acoustic energy. The second transducer layer is free of use for reception in response to the transmission from the second layer. Transmission with the second transducer layer is in response to an electrical signal different than the first electrical signal used for transmitting with the first transducer layer.

In a third aspect, an ultrasound transducer is provided for multi-purpose use. A first set of first elements have a first number of transducer layers. A second set of second elements have a different second number of transducer layers. The first and second elements are distributed along an azimuth dimension.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart of one embodiment of a method for using a same transducer for multiple purposes;

FIG. 2 is a graphical representation of one embodiment of a multi-layer transducer; and

FIG. 3 is a graphical representation of one embodiment of a multi-element transducer for multiple purpose uses.

### DETAILED DESCRIPTION OF THE DRAWINGS AND PRESENTLY PREFERRED EMBODIMENTS

Ultrasound transducers and associated connections with an ultrasound system are provided for multiple purpose ultrasound. For example, one type of element or one layer of an element in the transducer array is used for an imaging purpose, and another transducer layer or different type of element is used for another ultrasound purpose, such as application of acoustic radiation force as in remote palpitation, streaming, creating shear waves, moving microbubbles, or breaking microbubbles, or other energy transfer means like heat generation (useful for therapeutic purposes) and vibration (such as the compression and rarefaction, which is also useful for breaking microbubbles.) Each transducer layer or element is optimized for the respective purpose. The different elements or transducer layers are optimized for the desired transmit power, frequency, excitation pulse, or other purpose specific characteristic. Since the transducer includes both types of transducer layers or elements, a single transducer is provided for multiple ultrasound purposes.

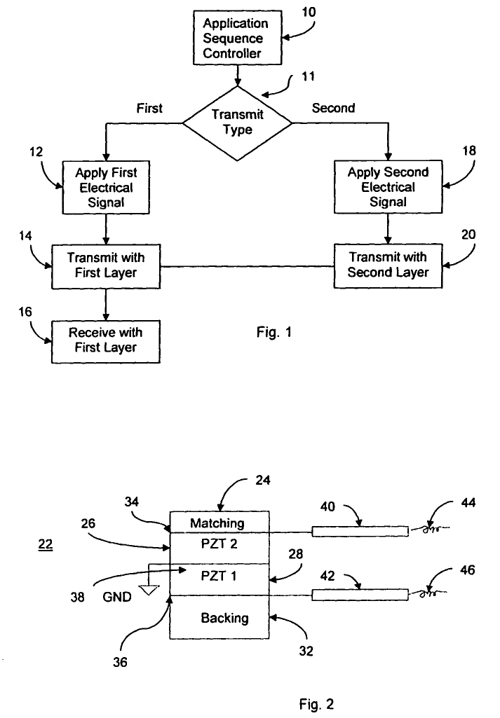

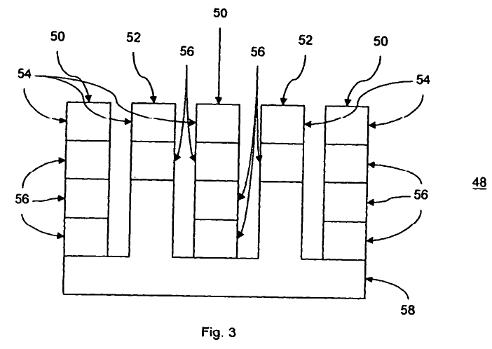

FIG. 1 shows one embodiment of a method for using a transducer to image and apply acoustic energy for other purposes. The method is implemented with a transducer disclosed in FIG. 2 or 3 or a different transducer. The acts shown in FIG. 1 may be performed in the same or different order. Acts 14 and 20 are shown as connected together to represent the use of a same transducer or probe for two different transmission purposes. The acts 14 and 20 are performed independently of each other, but may be performed in conjunction with each other such that transmission pursuant to act 14 or 20 contributes to operation of the respective acts 20 or 14. Addition, different or fewer acts may be provided.

In act 10, an application sequence with first and second signal types is chosen, decision point 11 routing first signal type to act 12, where an electrical signal is applied to a transducer element or transducer layer of an element. The electrical signal is at an ultrasound frequency or other frequency designed to generate ultrasound energy by transducing from the electrical signal. The electrical signal is a pulse wave or continuous wave signal. The signal is a square wave (either unipolar or bipolar), sinusoidal, or composed of any number of states and any number of cycles or partial cycles, such as a 1.5 cycle wave. Signals may be paired for pulse inversion or coded, as in Golay coded pulses. Signals may be broadband about a center frequency or chirped across a frequency band. Electrical signals for use in B-mode, flow mode, Doppler mode, color mode, spectral Doppler, M-mode or other imaging mode may be used.

For a given transmit event, such as associated with transmitting along one or more scan lines substantially simultaneously, the electrical signal is applied by routing through a direct connection or one or more switches from a transmitter or transmit channel to an element. A plurality of transmit channels and associated electrical signals are applied to different elements of the same array. The electrical signal is applied to a given element by connection with one or more electrodes on the element. The electrodes are positioned adjacent to transducer layers for transducing from the electrical signal to acoustic energy.

In act 14, an acoustic wave form is transmitted in response to the application of the electrical signal in act 12. Where the electrical signal is applied to one or more layers of transducer material in an element, the layers associated with the application generate acoustic energy. For example, a piezoelectric ceramic is caused to expand or contract in response to the electrical signal. As another example, a flexible membrane is caused to flex inward or outward from a chamber in response to the electrical signal. The electrical energy is transduced to acoustic energy in an ultrasound frequency, such as generating a waveform at 0.5 to 20 MHz.

In act 16, the same element and/or layer or layers of the element used for the transmission in act 14 receive acoustic echoes. Different transducers may alternatively or additionally receive the echoes. The echo signals are generated in response to the transmission of acoustic energy in act 14. The echoes cause compression or expansion of the transducer layer or flexing of a membrane of the transducer. In response to the acoustic echoes, the transducer transduces to an electrical energy.

The electrical energy generated in response to the echo signals is provided to a receiver or receive beamformer. The receive beamformer generates samples or signals representing given spatial locations scanned using a plurality of channels and associated elements of the transducer. Any of the imaging modes discussed herein may be used. The transducer element or transducer layer is optimized for imaging use, such as having a thickness, material composition, tuning, connection to transmit and receive beamformer channels, or other characteristic for ultrasound imaging.

Based on the sequence from act 10, the transmit type act 11 routes signals of a second type or purpose to act 18. In act 18, an electrical signal is applied to a same or different element as the electrical signal in act 12. For example, the electrical signal is applied to a different transducer layer of a same element. As another example, the electrical signal is applied to an adjacent or different element as the electrical signal of act 12.

The electrical signal applied in act 18 is the same or different as the electrical signal in act 12. For example, the electrical signal applied in act 18 has a greater amplitude, greater frequency, lesser frequency, longer duration, shorter duration, different type of waveform (e.g. square wave or sinusoidal), different number of cycles, or other characteristic different than the electrical signal applied in act 12. In one embodiment, the electrical signal has 100-300 volt peak-to-peak amplitude for act 18, and the electrical signal applied in act 12 has a lesser 99-200 volt peak-to-peak amplitude. As another example, the electrical signal applied in act 18 has a lower frequency, such as 1-2 MHz frequency, than the higher frequency signal applied in act 12, such as a 5 MHz signal. Thickness of each transducer layer, the signal generated and/or the relative tuning of the transducer layers may be used to provide electrical signals and associated acoustic waveforms in acts 12, 14 and 18, 20 for providing different center frequencies, such as a frequency of about three times center frequency used for another layer. Lesser or greater frequency separations may be provided.

In act 20, an acoustic waveform is transmitted in response to the application of the electrical signal in act 18. The transducer layer in the same element for acts 12 through 16 or a different element generate acoustic energy in response to application of the electrical signal.

The purpose of acts 18 and 20 is for other ultrasound uses than imaging. Accordingly, the application of the electrical signal in act 18 and the transmission of responsive acoustic energy in act 20 are performed only for transmission, and not for reception of acoustic echoes for imaging. The transducer layer or element used for acts 18 and 20 is free of use for reception even for imaging or just in response to the transmission of act 20. For example, a connection from a transducer layer used for act 20 connects with a transmitter or transmit beamformer without being connected to a receiver or receive beamformer.

The application of the electrical signal in act 18 and the responsive transmission in act 20 are performed for a single element or for a plurality of elements. For example, a plurality of elements received relatively delayed, apodized or both delayed and apodized electrical signals for generating a beam of acoustic energy. As another example, the same electrical signal is applied to a plurality of different elements. Different groups of elements may receive relatively delayed and/or apodized signals for focusing. As yet another example, the same electrical signal is applied to all elements in the transmit aperture or to just a single element without transmission by other elements. The application of electrical signal in act 12 is performed in response to transmit channels dedicated to specific elements. By using different transmit channels for different elements for imaging, a greater resolution is provided.

The acoustic energy generated in act 20 is for a non-imaging purpose. Any now known or later developed purpose may be implemented. For example, the acoustic waveform is used for acoustic radiation force or remote palpitation. The palpitation is imaged using the acts 12 through 16.

Act 20 may be used to create streaming of fluid. For example, U.S. Pat. No. 5,487,387, the disclosure of which is incorporated herein by reference, creates movement of fluids for identifying cysts. The acoustic energy is of sufficient intensity to initiate movement of any fluid located within a target lesion. Imaging is then performed of the target lesion to detect the presence or absence of fluid movement.

Another purpose is to create strain or other tissue movement through remote palpitation. Multiple applications of acoustic radiation in rapid succession cause strain or tissue palpitation. The associated displacement may then be imaged, such as disclosed in U.S. Pat. No. 6,371,912, the disclosure of which is incorporated herein by reference.

In another purpose, the acoustic energy is used to create shear waves, such as disclosed in U.S. Pat. No. 6,764,448, the disclosure of which is incorporated herein by reference. Acoustic energy is transmitted into a tissue in a given direction to provide a virtual extended shear wave. The shear wave generates an extended shear wave that propagates in a direction orthogonal to the original direction. The tissue responsive to the shear waves is imaged.

The acoustic energy may be used to create tissue movement for other purposes as well. Other purposes than tissue movement may be provided, such as applying acoustic energy for heating tissue, breaking structures, or other therapy. Another therapeutic use generates acoustic energy adapted to destroy or break microbubbles. The microbubbles contain drugs or other chemicals for delivery within a patient. By releasing the drugs through destruction of the microbubbles in specific locations, targeted drug delivery is provided. As yet another example of a different purpose for generating the acoustic energy in act 20, acoustic energy adapted to move microbubbles without breaking, such as through power or frequency selection, assists in the targeting of microbubbles. For example, microbubbles are caused to move adjacent to or perfuse into tissue using acoustic energy. Microbubbles may then be imaged or destroyed for release of drugs or destroyed and imaged to determine wash-in times or perfusion rates.

Acts 14 and 20 are performed sequentially. For example, act 20 is performed before act 14 for causing tissue movement or other purposes. Act 14 is then performed for imaging as a function of the tissue movement. In alternative embodiments, acts 14 and 20 are performed at substantially the same time. For example, the imaging associated with act 14 is provided at a same time as microbubbles are destroyed, palpitation is caused, or therapeutic ultrasound is applied in act 20. To avoid interference of imaging by the ultrasound energy applied for other purposes in act 20, the acoustic waveforms are applied at different frequencies or with different coding. The received acoustic energy for imaging is processed, such as filtering or coding, to identify or more greatly include information in response to the transmission in act 14 than the transmission in act 20.

FIG. 2 shows one embodiment of an element 24 of an ultrasound transducer 22 for multi-purpose use. The element 24 is part of a single element, 1D, or multidimensional transducer 22. The element 24 includes transducer layers 26 and 28 for implementing the method described above for FIG. 1. Other methods may be implemented using the transducer 22. The transducer element 24 includes two or more transducer layers 26, 28, one or more matching layers 30, a backing layer 32, electrodes 34, 36 and 38 for each of the transducer layers 26, 28, cables 40 and 42 connected with respective electrodes 34 and 36, and tuning circuits 44 and 46. Additional, different or fewer components may be provided. For example, a switch or multiplexer is provided for connecting a same cable 40, 42 or a same transmit channel to different ones of the transducer layers 26, 28 and associated electrodes 34, 36. The switch or multiplexer may be provided between the tuning circuits 44, 46 and the transmitter or along the cable 40, 42 where similar frequencies are to be used for the different purposes.

The two or more transducer layers 26, 28 are formed from a same or different transducer material. For example, a single crystal, such as PZN-PT or PMN-PT, is used. Other piezoelectric materials, such as PZT5H, or composite materials may be used. In another embodiment, one or more of the transducer layers 26, 28 are formed from a semiconductor material as a microelectromechanical or CMUT device. In one embodiment, the top transducer layer 26 is formed from a single crystal material or other material with a relatively lower acoustic impedance than a non-single crystal piezoelectric or other material used in the lower transducer layer 28. In yet another embodiment, one or more of the transducer layers 26, 28 are formed from an electrostrictive polymer.

The different transducer layers 26, 28 are optimized for different purposes. For example, the bottom transducer layer 28 is optimized for imaging or transmitting and receiving acoustic energy. The top transducer layer 26 is optimized for only transmission and not reception or for transmission of higher power or different frequencies than the other transducer layer 28. In one embodiment, the optimization is through type of material. In the example given above, the lower acoustic impedance transducer layer 26 used for other purposes provides a matching or acoustic impedance transition for the imaging transducer layer 28.

In another embodiment, the transducer layers 26, 28 are optimized for different frequency characteristics. For example, the imaging transducer layer 28 has a wider bandwidth or frequency response than the other transducer layer 26. As another example, the different transducer layers 26, 28 are optimized for use at different center frequencies. The thickness of the transducer layer 26, 28 controls frequency characteristic. The thickness of each transducer layer 26, 28 is selected to be about a quarter wavelength of the imaging frequency in one embodiment, but may have different thicknesses for use at different frequencies. The bottom layer 28 is in favor of imaging layer because it is easier to design for a wide bandwidth without interference of back reflection. The top layer 26 often shows a bi-mode feature in the spectrum because of the bouncing echo from the bottom transducer layer 28. The characteristics of the top layer 26 may be improved by electric tuning circuits 44 to achieve narrow band and high transmit efficiency at a desired frequency. The tuning circuits 44, 46 may be selected separately to optimize the center frequency and/or bandwidth of each layer for different applications. In one embodiment, the tuning circuits 44, 46 are inductors connected in series with a resistance provided by a transmit or receive circuitry and a capacitance provided by the element 24. The inductor values are different depending on the application. For example, a 22 pH inductor is provided for the circuit 44 for transmitting a 25 kilopascal per volt acoustic pressure at 1 MHz from the upper transducer layer 26, and a 2.2 μH inductor is provided for the circuit 46 to provide wideband pulse at 5 MHz for imaging with the transducer layer 28. Other tuning circuits the same or different for the different transducer layers 26, 28 may be used.

An alternative or additional characteristic of the element 24 for multi-purpose use is the connections provided by the cables 40 and 42. The cables 40 and 42 are coaxial, flex circuit, signal traces or other electrical conductors for connecting electrodes 34, 36, 38 and associated flex circuit material to circuitry of an ultrasound system. Since different transducer layers 26, 28 are intended for operation of different purposes, the respective cables 40, 42 connect with different circuitry. For example, the electrode 36 of the bottom transducer layer 28 is directly or indirectly connected by the cable 42 to a transmit and receive switch, a transmit beamformer and a receive beamformer. The cable 42 also indirectly connects to a detector, scan converter or other associated imaging circuits. The electrode 34 of the top transducer layer 26 used for just transmission of acoustic energy is connected by the cable 40 to a transmit beamformer through none or one or more switches. A connection to a receive beamformer is not provided.

In one embodiment, the cable 40 connecting the transducer layer 26 to a transmitter also connects other elements 24 to the same transmit channel. Signals from the same channel are applied to a plurality of different elements. The other cable 42 used for imaging connects the transducer layer 28 independently of other elements to a dedicated transmit and/or receive channels. For example, a 192 channel transmit beamformer is provided. 128 channels connect with 128 different elements for imaging. 64 channels each connect with two elements shorted together for non-imaging uses. The connections may be permanent, such as arranged within the transducer or flex circuits, or may be switchable through one or more multiplexers. In alternative embodiments, imaging is provided with two or more elements connected to a same transmit or receive channel, or other transmit purposes are implemented with dedicated transmit channels for each element 24.

Different ones of the two transducer layers 26, 28 are used for different purposes. For example, the top transducer layer 26 or the bottom transducer layer 28 is used for imaging, and the other transducer layer 28, 26 is used for pushing tissue, fluid or microbubbles, for breaking microbubbles or tissue structures, or for heating or other therapy. The different uses may dictate different design or optimization. For example, higher frequency imparts greater radiation force. For tissue palpitation or other uses, a transducer layer 26, 28 is adapted for higher frequency than used for imaging. As another example, microbubbles are more reactive to lower frequencies in some cases. Greater movement or even destruction may be provided with lower frequencies than used for imaging. Bandwidth or ability to operate pursuant to different amplitude levels may alternatively or additionally be used.

Different ones of the transducer layers 26, 28 may be used for different purposes at different times, such as using the upper transducer layer 26 for imaging in one circumstance and the lower transducer layer 28 for imaging in another circumstance, such as for imaging in different modes. For example, layer 26 may used for CW or color Doppler mode, while layer 28 is used for B-mode. Similarly, different transducer layers 26, 28 may be used for non-imaging purposes at different times, such as using an upper layer 26 adapted for higher frequency used for imaging at one time but remote palpitation at other times. Multiple transducer layers 26, 28 may be used for a common purpose. For example, both transducer layers 26, 28 are used to generate acoustic energy for non-imaging purposes. More than two transducer layers 26, 28 may be provided, such as providing a third transducer layer for implementing yet another purpose or for use with either of the other transducer layers 26, 28 for imaging or other purpose.

FIG. 3 shows another embodiment of a transducer 48 for multi-purpose use. The transducer is used for implementing the method described in FIG. 1 or a different method. FIG. 2 uses transducer layers in a same element distributed along a range dimension for different purposes. FIG. 3 shows different elements 50, 52 to be used for different purposes. The elements 50, 52 are distributed within the transducer 48 along an azimuth, elevation or both azimuth and elevation dimensions. For azimuth distribution, the transducer 48 is a linear, curved linear or other one-dimensional phased array. In alternative embodiments, the array 48 is provided within a multi-dimensional array, such as within a 1.5D or 2D array.

Each type of element 50, 52 is optimized for a different use or purpose. The optimization is provided by a different number of transducer layers 56. As shown in FIG. 3, one type of element 50 has three transducer layers 56 between one or more matching layers 54 and a backing layer 58. The other type of element 52 has a single transducer layer 56 between the matching layers 54 and backing layer 58. In alternative embodiments, one or both of the types of elements 50, 52 have a different number of transducer layers 56. The number of layers may correspond to the desired center frequency of use. For example, a 1 MHz and 10 MHz dual frequency transducer is provided. 10 MHz operation is provided by the elements 52 with a single transducer layer 56. The 1 MHz operation is provided with the elements 50 with 5 to 7 layers of transducer material 56. By varying the number of layers, a large frequency separation may be provided, such as a frequency of one type of element being at least about five times the center frequency of operation of another type of element. Additional types of elements that differ in number of transducer layers or other characteristics, such as thickness or poling of one or more transducer layers, may be provided.

As shown in FIG. 3, the different types of elements 50, 52 are provided as every other element, but groups of one type of element may separate one or more elements of another type. The element pitch is half lambda of the lowest frequency to be used, such as the imaging or frequency used for other purposes. For example, a 150 μm pitch is used for 5 MHz imaging.

Any now known or later developed manufacturing technique may be used for the transducer array 48. For example, two layers of PZT material are stacked for every other element 50, 52. The stacked partial number of layers is then interconnected by filling with backing or other lossy material. This multi-layer composite has a kerf width or width between PZT materials equal to or slightly larger than an element width. The multi-layer composite is then bonded to another PZT layer. One or more matching layers 54 and the backing block 58 are stacked on the top and bottom of the composite. The structure is then diced to form acoustically and electrically isolated elements. Some of the elements have a single layer and other elements have three layers of transducer material 56.

In one embodiment, the different elements 50, 52 independently connect to different circuitry, such as different transmit channels. Elements used for purposes other than imaging may be free of connection to receiver or receive beamformer channels. In one embodiment, the elements 50, 52 used for purposes other than imaging are connected together for transmitting acoustic energy in one or more groups. For example, two or more of the elements 50 with multiple or a greater number of transducer layers 56 are connected in common or shorted together to a same transmit channel to reduce the channel count. Different groups of the elements 50 are connected to different transmit channels. The other elements 52 used for imaging independently connect to separate transmit and receive beamformer channels.

As a 1.25 or 1.5 dimensional array, the center array provides both low and high frequency alternating elements 50, 52. The outer rows of elements provide for low frequency use. The greater transmit aperture for low frequency use allows for a greater application of force, such as for palpitation of tissue. The multiple rows provide focal depth and focal size control along elevation for high power acoustic energy to be at desired locations. Different voltage levels may be applied to elements of the center row as compared to the outer rows to provide a more uniform pressure field. The outer rows may be either multilayer or single layer transducers.

The element 24 of FIG. 2 or one or more of the elements 50, 52 of FIG. 3 has a concave surface along an elevation dimension in one embodiment. A parabolic or generally curved concave surface to provide focus and associated low loss urethane lens may increase high frequency sensitivity and bandwidth. High frequency signals excite only the center portion of the elevation aperture, resulting in a narrower beam for imaging resolution. For multi-layers of transducer material, the top layer is concave. Other layers may be flat or concave. In alternative embodiments, all the layers are flat.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. An ultrasound transducer for multi-purpose use, the transducer comprising:
a first transducer layer of an element, the first transducer layer operable for transmission and reception;
a second transducer layer of the element, the first and second transducer layers distributed in range, the second transducer layer operable only for transmission and not for reception;
a first cable connected with a first electrode of the first transducer layer; and
a second cable connected with a second electrode of the second transducer layer.

2. The ultrasound transducer of claim 1 further comprising a matching layer adjacent the second transducer layer and a backing layer adjacent the first transducer layer.

3. The ultrasound transducer of claim 1 wherein the second transducer layer is operable for pushing.

4. The ultrasound transducer of claim 1 wherein the second transducer layer is operable for breaking.

5. The ultrasound transducer of claim 1 wherein the second transducer layer is operable for heating.

6. The ultrasound transducer of claim 1 wherein the second transducer layer is operable for a narrower band of frequencies than the first transducer layer.

7. The ultrasound transducer of claim 1 further comprising:
a first tuning circuit connected with the first transducer layer and first cable; and
a second tuning circuit connected with the second transducer layer and second cable, the first tuning circuit operable different frequencies of operation than the second tuning circuit.

8. The ultrasound transducer of claim 1 wherein the second transducer layer comprises a single crystal piezoelectric material and the first transducer layer comprises a different material than the second transducer layer.

9. The ultrasound transducer of claim 1 wherein the second cable connects with the second electrode and at least an additional electrode of an additional element and wherein the first cable connects with only the element.

10. A method for using a transducer to image and apply other acoustic energy, the method comprising:
transmitting with a first transducer layer of an element of the transducer, the transmitting being in response to a first electrical signal;
receiving with the first transducer layer of the element of the transducer, the receiving being in response to the transmitting; and
transmitting with a second transducer layer of the element of the transducer, the second transducer layer free of use for reception in response to the transmitting with the second transducer layer, the first and second transducer layers distributed in range, the transmitting with the second transducer layer being in response to a second electrical signal different than the first electrical signal.

11. The method of claim 10 wherein transmitting with the first transducer layer and receiving comprise imaging with the first transducer layer.

12. The method of claim 10 wherein transmitting with the second transducer layer comprises generating acoustic energy to create shear waves.

13. The method of claim 10 wherein transmitting with the second transducer layer comprises generating acoustic energy to move microbubbles.

14. The method of claim 10 wherein transmitting with the second transducer layer comprises generating acoustic energy to break microbubbles.

15. The method of claim 10 wherein transmitting with the second transducer layer comprises generating acoustic energy to heat tissue.

16. The method of claim 10 wherein transmitting with the second transducer layer comprises generating acoustic energy to create streaming of fluid.

17. The method of claim 10 wherein transmitting with the second transducer layer comprises generating acoustic energy to create tissue movement.

18. The method of claim 10 further comprising:
sequentially performing the transmitting with the first transducer layer and transmitting with the second transducer layer.

19. The method of claim 10 further comprising:
performing the transmitting with the first transducer layer and transmitting with the second transducer layer at substantially a same time, the first electrical signal being at a different frequency than the second electrical signal.

20. The method of claim 10 further comprising:
tuning the first transducer layer at a different frequency than the second transducer layer.

21. The method of claim 20 wherein the tuning comprises tuning the second transducer layer for a second center frequency at least about 3 times a first center frequency for the first transducer layer.

22. The method of claim 10 wherein transmitting in response to the first and second electrical signals comprises transmitting in response to the first electrical signal having different power than the second electrical signal.

23. The method of claim 10 wherein transmitting with the first transducer layer comprises transmitting with a first type of transducer material and transmitting with the second transducer layer comprises transmitting with a different, second type of transducer material.

24. The method of claim 10 further comprising:
transmitting with at least an additional element in response to the second electrical signal, the element and the additional element connected in common with a cable;
wherein transmitting in response to the first electrical signal comprises transmitting with a transmit channel dedicated to the element and wherein receiving comprises receiving with a receive channel dedicated to the element.

25. An ultrasound transducer for multi-purpose use, the transducer comprising:
a first set of first elements having a first number of transducer layers; and
a second set of second elements having a different, second number of transducer layers, the first and second elements distributed along an azimuth dimension;
wherein the distribution of the first and second elements is every other element being one of the first elements and the other elements being one of the second elements.

26. The ultrasound transducer of claim 25 wherein the first number is one and the second number is three.

27. The ultrasound transducer of claim 25 wherein the first elements are operable at a first center frequency and the second elements are operable at a different, second center frequency.

28. The ultrasound transducer of claim 27 wherein the first center frequency is at least about five times the second center frequency.

29. The ultrasound transducer of claim 25 wherein groups of the second elements connect with first separate transmit channels and each of the first elements connect with second separate transmit channels.

30. The ultrasound transducer of claim 25 wherein the second elements comprise at least one transducer layer having a concave surface along an elevation dimension.
